(19) United States
(12) Patent Application Publication
(10) Pub. No.: US 2010/0152507 A1
(43) Pub. Date: Jun. 17, 2010

(54) Process for Using Layered Sphere Catalysts with High Accessibility Indexes

(76) Inventors: Gregory J. Gajda, Mount Prospect, IL (US); Bryan K. Glover, Algonquin, IL (US); Antoine Negiz, Wilmette, IL (US); Mark G. Riley, Hinsdale, IL (US); John J. Senetar, Naperville, IL (US); Erik M. Holmgreen, Racine, WI (US)

Correspondence Address: HONEYWELL/UOP;PATENT SERVICES, 101 COLUMBIA DRIVE, P O BOX 2245 MAIL STOP AB/2B, MORRISTOWN, NJ, 07962, US

(21) Appl. No.: 12/335,639

(22) Filed: Dec. 16, 2008

(51) Int. Cl. C07C 5/05

(52) U.S. Cl. 585/275

(57) ABSTRACT

A process and catalyst for use in the selective hydrogenation of acetylene to ethylene is presented. The catalyst comprises a layered structure, wherein the catalyst has an inner core and an outer layer of active material. The catalyst further includes a metal deposited on the outer layer, and the catalyst is formed such that the catalyst has an accessibility index between 3 and 500.

### FIELD OF THE INVENTION

This invention relates to a layered catalyst composition, a process for preparing the composition and hydrocarbon conversion processes using the composition. The layered composition comprises an inner core, and an outer layer, comprising an inorganic oxide, bonded to the inner core.

### BACKGROUND OF THE INVENTION

Platinum based catalysts are used for numerous hydrocarbon conversion processes. In many applications promoters and modifiers are also used. One such hydrocarbon conversion process is the dehydrogenation of hydrocarbons, particularly alkanes such as isobutane, which are converted to isobutylene. For example, U.S. Pat. No. 3,878,131 (and related U.S. Pat. No. 3,632,503 and U.S. Pat. No. 3,755,481) discloses a catalyst comprising a platinum metal, a tin oxide component and a germanium oxide component. All components are uniformly dispersed throughout the alumina support. U.S. Pat. No. 3,761,531 (and related U.S. Pat. No. 3,682,838) discloses a catalytic composite comprising a platinum group component, a Group IV A metallic component, e.g., germanium, a Group VA metallic component, e.g., arsenic, antimony and an alkali or alkaline earth component all dispersed on an alumina carrier material. Again all the components are evenly distributed on the carrier.

U.S. Pat. No. 3,558,477, U.S. Pat. No. 3,562,147, U.S. Pat. No. 3,584,060 and U.S. Pat. No. 3,649,566 all disclose catalytic composites comprising a platinum group component and a rhenium component on a refractory oxide support. However, as before, these references disclose that the best results are achieved when the platinum group component and rhenium component are uniformly distributed throughout the catalyst.

It is also known that for certain processes selectivity towards desirable products is inhibited by excessive residence time of the feed or the products at the active sites of the catalyst. Thus, U.S. Pat. No. 4,716,143 describes a catalyst in which the platinum group metal is deposited in an outer layer (about 400 μm) of the support. No preference is given to how the modifier metal should be distributed throughout the support. Similarly U.S. Pat. No. 4,786,625 discloses a catalyst in which the platinum is deposited on the surface of the support whereas the modifier metal is evenly distributed throughout the support.

U.S. Pat. No. 3,897,368 describes a method for the production of a noble metal catalyst where the noble metal is platinum and the platinum is deposited selectively upon the external surface of the catalyst. However, this disclosure describes the advantages of impregnating only platinum on the exterior layer and utilizes a specific type of surfactant to achieve the surface impregnation of the noble metal.

The art also discloses several references where a catalyst contains an inner core and an outer layer or shell. For example, U.S. Pat. No. 3,145,183 discloses spheres having an impervious center and a porous shell. Although it is disclosed that the impervious center can be small, the overall diameter is ⅛″ or larger. It is stated that for smaller diameter spheres (less than ⅛″), uniformity is hard to control. U.S. Pat. No. 5,516,740 discloses a thin outer shell of catalytic material bonded to an inner core of catalytically inert material. The outer core can have catalytic metals such as platinum dispersed on it. The '740 patent further discloses that this catalyst is used in an isomerization process. Finally, the outer layer material contains the catalytic metal prior to it being coated onto the inner core.

U.S. Pat. No. 4,077,912 and U.S. Pat. No. 4,255,253 disclose a catalyst having a base support having deposited thereon a layer of a catalytic metal oxide or a combination of a catalytic metal oxide and an oxide support. WO98/14274 discloses a catalyst which comprises a catalytically inert core material on which is deposited and bonded a thin shell of material containing active sites.

The present invention provides for improved activity and selectivity with respect to selective hydrogenation of acetylene compounds.

### SUMMARY OF THE INVENTION

The present invention provides for the selective hydrogenation of acetylene to ethylene. The process comprises contacting a feedstream comprising ethylene and acetylene with a new catalyst thereby producing an output ethylene rich stream with a reduced acetylene content. The catalyst comprises a layered catalyst having an inner core made of an inert material. An outer layer is bonded to the inner core, where the outer layer comprises a metal oxide. On the outer layer a first catalytic metal and a second catalytic metal are deposited, where the first metal is selected from an IUPAC Group 8 to 10 metal, and the second metal is selected from an IUPAC Group 11 or Group 14 metal. The materials for the layered catalyst are chosen, and assembled onto the catalyst wherein the catalyst has an accessibility index between 3 and 500, or a void space index (VSI) between 0 and 1, or both an AI between 3 and 500 and a VSI between 0 and 1.

In another embodiment the process comprises passing a feedstream through a demethanizer, thereby creating a demethanized ethylene stream. The demethanized stream will also have a reduced carbon monoxide content. The demethanized ethylene stream comprising ethylene and acetylene is contacted with a new catalyst thereby producing an output ethylene rich stream with a reduced acetylene content. The catalyst comprises a layered catalyst having an inner core made of an inert material. An outer layer is bonded to the inner core, where the outer layer comprises a metal oxide. On the outer layer a first catalytic metal and a second catalytic metal are deposited, where the first metal is selected from an IUPAC Group 8 to 10 metal, and the second metal is selected from an IUPAC Group 11 or Group 14 metal. The materials for the layered catalyst are chosen, and assembled onto the catalyst wherein the catalyst has an accessibility index between 3 and 500, or a void space index (VSI) between 0 and 1, or both an AI between 3 and 500 and a VSI between 0 and 1.

Other objects, advantages and applications of the present invention will become apparent to those skilled in the art from the following detailed description and Figures.

### BRIEF DESCRIPTION OF THE DRAWINGS

FIG. 1 is a diagram of the front end use of the catalyst for hydrogenation of acetylene; and

FIG. 2 is a diagram of the tail end use of the catalyst for hydrogenation of acetylene.

### DETAILED DESCRIPTION OF THE INVENTION

Ethylene and propylene, light olefin hydrocarbons with two or three carbon atoms per molecule, respectively, are important chemicals for use in the production of other useful materials, such as polyethylene and polypropylene. Polyethylene and polypropylene are two of the most common plastics found in use today and have a wide variety of uses for both as a material fabrication and as a material for packaging. Other uses for ethylene and propylene include the production of vinyl chloride, ethylene oxide, ethylbenzene and alcohol. Steam cracking or pyrolysis of hydrocarbons produces most of the ethylene and some propylene. Ethylene is produced through several means, such as steam cracking of hydrocarbons, catalytic cracking of hydrocarbons, or olefin cracking of larger olefinic feedstocks. However, ethylene for use in the production of polyethylene needs to be substantially pure. The methods of producing ethylene generate a product stream with substantial amount of acetylene, which can be as high as 2 to 3 volume percent of the ethylene/ethane stream.

The selective hydrogenation of acetylene improves the quality of the ethylene product stream while increasing the amount of ethylene is achieved by using a more selective catalyst. The catalyst in the present invention comprises a material having properties that distinguish it from current commercial catalysts. These properties can be determined from the activity indexes for choosing a catalyst that has good selectivity in this process. The catalyst selectively hydrogenates the acetylene to an amount less than 5 ppm of the ethylene product stream, and will preferable reduce the acetylene to less than 1 ppm.

The catalyst is a layered catalyst having an inner core comprising an inert material. An outer layer is bonded to the inner core, wherein the outer layer comprises a metal oxide. The catalyst includes a first metal selected from an IUPAC Group 8-10 metal which is deposited on the outer layer, and a second metal selected from an IUPAC Group 11 or 14 metal which is deposited on the outer layer. The catalyst also has an accessibility index (AI) of between 3 and 500, with a preferred accessibility index between 3 and 20, and a more preferred accessibility index between 4 and 20. The accessibility index is equal to the surface area of the outer layer times the diameter of the particles times 100 divided by the effective thickness of the layer in micrometers, or cm²/(g), but where the surface area is from only the outer layer, the entire particle weight is taken into account.

The first metal deposited on the outer layer is preferably platinum or palladium or a mixture thereof, and is deposited in a concentration between 100 and 50,000 ppm wt. of the catalyst. Preferably the first metal is deposited in a concentration between 200 and 20,000 ppm wt. of the catalyst.

The second metal deposited on the outer layer is preferably one or more of the metals including copper, silver, gold, tin, germanium, and lead. The second metal is deposited on the outer layer in an amount such that the atomic ratio of the first metal to the second metal is between 0.1 and 10.

The catalyst inner core comprises an inert material, made up of one or more of the following: cordierite, mullite, olivine, zirconia, spinel, kyanite, aluminas, silicas, aluminates, silicates, titania, nitrides, carbides, borosilicates, boria, aluminum silicates, magnesia, fosterite, kaolin, kaolinite, montmorillonite, saponite, bentonite, clays that have little or low acidic activity, gamma alumina, delta alumina, eta alumina, and theta alumina. The inner core has an effective diameter of between 0.05 mm and 10 mm, and preferably from about 0.8 mm to about 5 mm and more preferably from about 0.8 mm to about 3 mm. By effective diameter it is meant, for non-spherical shapes, the diameter that the shaped particle would have if it were molded into a sphere. In a preferred embodiment, the dried shaped particles are substantially spherical in shape.

The outer layer is deposited on and bonded to the inner core to an effective thickness between 1 and 200 micrometers. A preferred outer layer thickness is between 20 and 100 micrometers, with a more preferred outer layer thickness between 20 and 70 micrometers. The actual thickness will vary somewhat around the particle. The term effective thickness is intended to mean the thickness based upon a layer if the material were uniformly distributed over the surface of the inner core. The inner core will have an irregular surface and this can lead to some irregularities in the distribution of the material of the outer layer. The material of the outer layer is selected from one or more of the following: gamma alumina, delta alumina, eta alumina, theta alumina, silica-alumina, zeolites, nonzeolitic molecular sieves, titania, and zirconia.

In an alternative embodiment, the catalyst is a layered catalyst having an inner core comprising an inert material. An outer layer is bonded to the inner core, wherein the outer layer comprises a metal oxide. The catalyst includes a first metal selected from an IUPAC Group 8-10 metal which is deposited on the outer layer, and a second metal selected from an IUPAC Group 11 or 14 metal which is deposited on the outer layer. The catalyst also has a void space index (VSI) of between 0 and 1, with a preferred void space index between 0.0001 and 0.5, and a more preferred void space index between 0.001 and 0.3. The void space index is equal to the pore volume times the average pore radius of the outer layer times the diameter of the particle and divided by the effective thickness of the outer layer, or in units of cm³*μm/g. The pore volume is the pore volume of the outer layer, whereas the weight of the whole catalyst is taken into account, and not just the weight of the outer layer.

The inert inner core is selected from the materials as mentioned above, and the outer layer comprises a material from the list above. The first and second metals deposited on the outer layer are selected from the metals listed above for the first and second metals.

Control of the selective hydrogenation process is important to minimize the hydrogenation of ethylene, thereby losing some of the product, and this control can be improved by selecting catalysts having an AI greater than 3, or a VSI less than 1, or both.

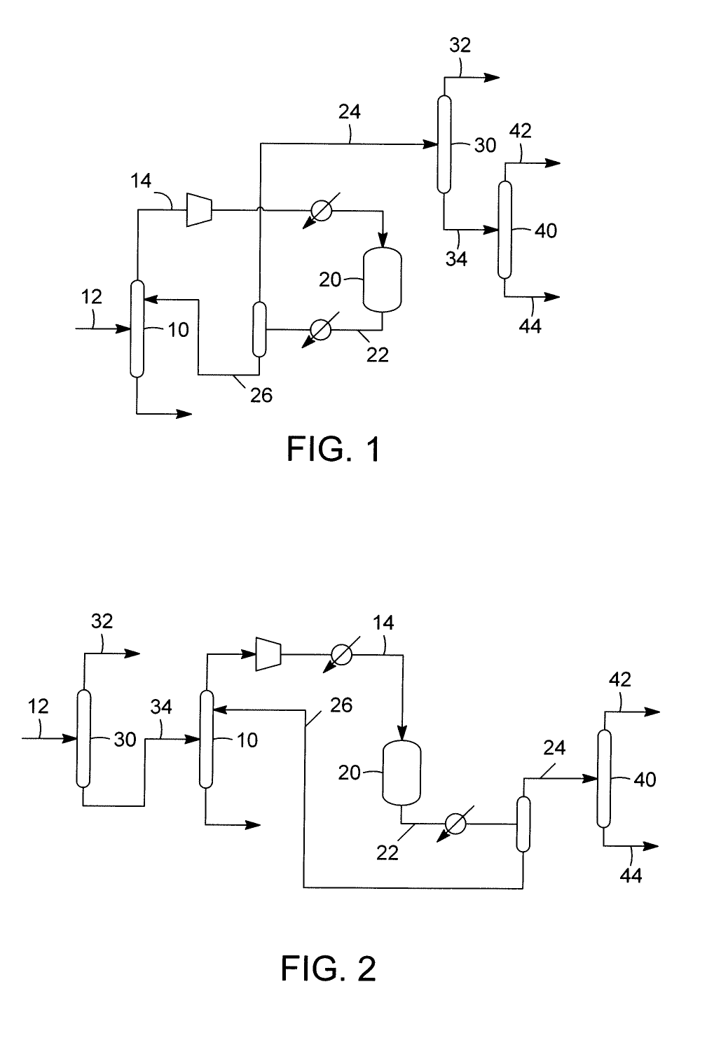

This catalyst is useful for the selective hydrogenation of acetylene to ethylene, while having minimal side reactions such as hydrogenation of the ethylene to ethane. The process is shown in FIG. 1, or a front end process. First a process feedstream 12 comprising ethylene, ethane and acetylene is passed through a deethanizer 10, and the overhead ethylene rich stream 14 is passed to the selective hydrogenation reactor 20. Typically the ethylene rich stream 14 is compressed and temperature adjusted before passing to the selective hydrogenation reactor 20. In general, temperature adjusting will be cooling the ethylene rich stream 14 that has been compressed. The process for using the catalyst comprises contacting the overhead feedstream 14 having ethylene and acetylene with the catalyst having either an AI between 3 and 500, or a VSI between 0 and 1, or both an AI between 3 and 500 and a VSI between 0 and 1, at reaction conditions, thereby creating an ethylene output stream, wherein the catalyst is as described above. The selective hydrogenation reaction conditions include pressures between 100 kPa and 14.0 MPa, with preferred pressures between 500 kPa and 10.0 MPa, and with more preferred pressures between 800 kPa and 7.0 MPa. The temperatures for the selective hydrogenation are between 10° C. to 300° C., with preferred temperatures between 30° C. to 200° C.

The selective hydrogenation conditions include a hydrogen to acetylene molar ratio between 0.1 and 10,000, but a preferred molar ratio between 0.1 and 10. The molar ratio is more preferred to be between 0.5 and 5, and with a most preferred ratio between 0.5 and 3. The source of the process feedstream 12 can be from a catalytic naphtha cracker, and in the process of producing an ethylene rich feedstream, a significant amount of carbon monoxide is generated. The amount of carbon monoxide can be between 1 and 8000 ppm by volume. When there is a high amount of carbon monoxide, the monoxide acts as a reversible blocker to active catalyst sites. The operating conditions of the selective hydrogenation reactor can include a gas hourly space velocity (GHSV) of between 1,000 and 15,000 hr⁻¹, and preferably a gas hourly space velocity of between 2,000 and 12,000 hr⁻¹. In a most preferred operation, the GHSV is between 8,000 and 12,000 hr⁻¹.

The selective hydrogenation reactor 20 passes an output stream 22 having a reduced acetylene content. The output stream 22 is cooled and will generate some condensate. The output stream 22 is separated into a condensate stream 26 which is passed back to the deethanizer 10 as reflux, and into a vapor stream 24. The vapor stream 24 is passed to a demethanizer 30 where the vapor stream 24 is split into a methane rich stream 32 which includes hydrogen and residual carbon monoxide, and an ethane/ethylene stream 34. The ethane/ethylene stream 34 is passed to an ethane/ethylene splitter 40 for separating out the ethane from the ethylene. An overhead stream 42 comprising ethylene is generated at a quality level for use as a polymer feedstock. A bottoms stream 44 comprising ethane is directed to other processing units, or as an end product.

In another embodiment, the process for selective hydrogenation of acetylene to ethylene is shown in FIG. 2, or a tail end process. First a process feedstream 12 is passed through a demethanizer 30, creating an overhead stream 32 comprising methane and carbon monoxide, and a demethanizer bottoms stream 34 comprising ethane, ethylene, acetylene and C3+ hydrocarbons. The demethanizer bottoms stream 34 is passed to a deethanizer 10 where the deethanizer splits the demethanizer bottoms stream into a deethanizer overhead, or ethylene, stream 14 comprising ethane, ethylene, and acetylene, and a bottoms stream comprising the C3+ hydrocarbons. The deethanizer overhead stream 14 is passed to a selective hydrogenation reactor 20 where the acetylene is selectively converted to ethylene. The overhead stream 14 can be compressed and temperature adjusted before passing to the selective hydrogenation reactor 20. In general, temperature adjusting is the cooling of the overhead stream 14 that is heated due to compression. The selective hydrogenation feed may include an additional hydrogen feedstream as needed. The ethylene stream 14 is contacted with a selective hydrogenation catalyst, having either an AI between 3 and 500, or a VSI between 0 and 1, or both, within the reactor at reaction conditions, wherein the catalyst is as described above.

The selective hydrogenation reaction conditions include pressures between 100 kPa and 14.0 MPa, with preferred pressures between 500 kPa and 10.0 MPa, and with more preferred pressures between 800 kPa and 7.0 MPa. The temperatures for the selective hydrogenation are between 10° C. to 300° C., with preferred temperatures between 30° C. to 200° C. The hydrogen to acetylene molar ratio is between 0.1 and 20, but a preferred molar ratio between 0.1 and 10. The molar ratio is more preferred to be between 0.5 and 5, and with a most preferred ratio between 0.5 and 3. The source of the process feedstream 12 can be from a catalytic naphtha cracker, steam cracker, or olefin cracking unit, and in the process of producing an ethylene rich feedstream, a significant amount of carbon monoxide is generated. However, with the feedstream passing through the demethanizer 30 before passing to the selective hydrogenation reactor 20, the amount of carbon monoxide can be between 0.1 and 10 ppm by volume. The operating conditions of the selective hydrogenation reactor can include a gas hourly space velocity (GHSV) of between 1,000 and 5,000 hr⁻¹, with a preferred GHSV below 4,000 hr⁻¹.

The selective hydrogenation reactor 20 generates a product stream 22 with a reduced acetylene content, and is passed to an ethane/ethylene splitter 40. The product stream 22 is cooled and will generate some condensate. The product stream 22 is passed to a vapor-liquid separator where the condensate 26 is recovered and passed back to the deethanizer 10 as reflux. The vapor stream 24 is passed to the splitter, where the splitter 40 generates an overhead stream 42 comprising ethylene is generated at a quality level for use as a polymer feedstock and a bottoms stream 44 comprising ethane is directed to other processing units, or as an end product.

The catalyst for use in the tail end process, that has the methane and a portion of the carbon monoxide removed before the selective hydrogenation, can be treated with an alkali metal to reduce the acidity of the catalyst. The catalyst is treated with an alkali metal in an amount less than 0.5 wt % of the outer layer, and preferably between 0.1 wt % and 0.5 wt % of the outer layer. Alkali metals useful include lithium (Li), sodium (Na), potassium (K), rubidium (Rb), and cesium (Cs). While treating with an alkali metal, it is molar amounts that give comparable activity, i.e. an atom of Li gives the same response as an atom of K. Therefore, the weight amounts for the lighter lithium is reduced according to the ratio of the atomic weights. For example, with Pd-only and Pd/Ag catalysts, 3300 ppm wt. K and 500 ppm wt Li have similar activities and selectivities.

However, for front end catalysts the addition of an alkali metal indicates increased activity, but decreased selectivity. For catalysts tested having Pd only on the outer layer, lower potassium gives higher activity and selectivity, or lower ethane formation. This shows preferential acetylene hydrogenation over ethylene hydrogenation, and lithium gives higher activity, but lower selectivity. For catalysts tested having Pd/Ag on the outer layer, lower potassium also gives higher activity and lower selectivity.

Table 1 compares the layered catalysts of the present invention having layer thicknesses from 5 to 200 micrometers of either gamma or theta alumina with a conventional catalyst prepared on an alpha alumina and where the conventional catalyst has its surface-impregnated to various depths from 25-300 um. All catalysts are taken to be 3 mm spheres for a common basis of presentation. The parameters indicate why very thin active zones are not practical for conventional catalysts. The active zones are defined as the region in which at least 90% of the active metal/active sites occur. Typical loadings become very high % monolayer coverage which yield poor metal utilization and often have very large metal particle agglomerates. Particularly distinguishing parameters are the surface area*particle diameter*100/active zone thickness (cm²/g), or AI, and pore volume*average pore radius*particle diameter/thickness (cm³*μm/g), or VSI.

TABLE 1
Activity Indexes

| Active zone | active layer | Void Space | Accessibility |
|---|---|---|---|
| material | thickness (μm) | Index (VSI) | Index (AI) |
| gamma alumina | 5 | 0.0562 | 11.94 |
| gamma alumina | 12.5 | 0.0282 | 11.85 |
| gamma alumina | 25 | 0.0154 | 11.71 |
| gamma alumina | 50 | 0.00815 | 11.43 |
| gamma alumina | 100 | 0.00424 | 10.91 |
| gamma alumina | 200 | 0.00222 | 10.02 |
| theta alumina | 5 | 0.135 | 5.37 |
| theta alumina | 12.5 | 0.0791 | 5.33 |
| theta alumina | 25 | 0.0469 | 5.27 |
| theta alumina | 50 | 0.0260 | 5.14 |
| theta alumina | 100 | 0.0139 | 4.91 |
| theta alumina | 200 | 0.00738 | 4.51 |
| alpha alumina | 25 | 21.22 | 0.293 |
| alpha alumina | 50 | 20.72 | 0.286 |
| alpha alumina | 100 | 19.78 | 0.273 |
| alpha alumina | 200 | 18.16 | 0.250 |
| alpha alumina | 300 | 16.80 | 0.232 |

The present invention used gamma and theta alumina for the outer layer of the catalyst, and had various effective thicknesses. The catalyst of the present invention has a high accessibility index, greater than 3, and a low void space index, less than 1, relative to a standard commercial catalyst using alpha alumina as the outer coating. Conventional catalysts using alpha alumina have very large average pore diameters. The indexes indicate why thin active zones are not practical for conventional catalysts. The active zones are regions in which >90% of the active metal sites occur. Conventional catalysts yield poor metal utilization because, with thin active zones, they have very high percent monolayer coverages and large metal particle agglomerates. Changing the pore size of the catalyst improves the performance of the selective hydrogenation for the front end process.

From the tests, catalyst activity tends to increase for catalysts with outer layer effective thicknesses in the range of 5 to 50 micrometers. This suggests thinner layers will give better performance. The catalysts of the present invention allow for thinner layers with lower metal deposition. This has the potential to reduce the tendency to accumulate heavy by-products and thereby reduce the deactivation of the catalyst.

### Catalyst Preparation Procedure:

The catalyst is prepared by adding a solution of the appropriate metal salts to the desired amount of support. The appropriate metal salts are typically nitrates. In particular, a 1% HNO3 solution, relative to the support weight, is diluted with deionized water to provide a volume of solution approximately equivalent to the support volume, or a 1:1 solution to support volume ratio. The solution is contacted with the support at room temperature for one hour with constant agitation, or rolling to insure good support and solution contact. The solution is then heated to 100° C. and the liquid evaporated over a period of time that is greater than 3 hours, thereby created the impregnated support. The final support should be ‘free-rolling’ or freely moving in the container. The final moisture content will vary with the specific support, but is typically in the range of 20 to 30 wt %.

The impregnated support is then transferred to a container suitable for calcination and reduction. The support is dried at 120° C. in flowing dry air for 3 hours, then ramped up to 450° C. in flowing dry air at a rate of 5° C./min and held at 450° C. for 1 hour. The sample is cooled to room temperature.

For reduction, the sample is ramped to 200° C. in flowing dry N2 at a rate of 5° C./min, and held at 200° C. for one hour. The flowing dry N2 is shut off and hydrogen is then flowed over the catalyst and held for 3 hours. The hydrogen is then switched to nitrogen and the catalyst sample is cooled to room temperature.

For a two step procedure, the calcined and reduced catalyst from the first step is used as the support for the second step and the typical impregnation, drying, calcination and reductions steps followed with the second set of metal salts in solution.

While the invention has been described with what are presently considered the preferred embodiments, it is to be understood that the invention is not limited to the disclosed embodiments, but it is intended to cover various modifications and equivalent arrangements included within the scope of the appended claims.

1. A process for the selective hydrogenation of acetylene to ethylene, comprising:
contacting a feedstream comprising ethylene and acetylene with a catalyst at reaction conditions thereby creating an output stream with a reduced amount of acetylene, wherein the catalyst comprises:
a layered catalyst having an inner core comprising an inert material;
an outer layer bonded to the inner core, wherein the outer layer comprises a metal oxide;
a first metal deposited on the outer layer, wherein the first metal is an IUPAC Group 8-10 metal; and
a second metal deposited on the outer layer, wherein the second metal is an IUPAC Group 11 or Group 14 metal;
wherein the catalyst has an accessibility index (AI) between 3 and 500, or a void space index (VSI) between 0 and 1, or both an AI between 3 and 500 and a VSI between 0 and 1.

2. The process of claim 1 wherein the selective hydrogenation conditions include pressures between 100 kPa and 14.0 MPa.

3. The process of claim 2 wherein the selective hydrogenation conditions include pressures between 500 kPa and 10.0 MPa.

4. The process of claim 3 wherein the selective hydrogenation conditions include pressures between 800 kPa and 7.0 MPa.

5. The process of claim 1 wherein the selective hydrogenation conditions include a temperature of 10° C. to 300° C.

6. The process of claim 5 wherein the selective hydrogenation conditions include a temperature of 30° C. to 200° C.

7. The process of claim 1 wherein the selective hydrogenation conditions include a hydrogen to acetylene molar ratio between 0.1 and 10000.

8. The process of claim 1 wherein the feedstream includes amounts of carbon monoxide (CO) between 1 and 8000 ppm by volume.

9. The process of claim 1 further comprising passing the ethylene output stream to a demethanizer, thereby creating a methane rich stream, and an ethane/ethylene stream.

10. The process of claim 9 further comprising passing the ethane/ethylene stream to an ethane/ethylene splitter, thereby creating an ethylene product stream.

11. A process for selective hydrogenation of acetylene to ethylene, comprising:
passing an ethylene stream through a demethanizer, thereby creating a demethanized ethylene stream;
contacting the demethanized ethylene stream with a catalyst at reaction conditions, wherein the catalyst comprises:
a layered catalyst having an inner core comprising an inert material;
an outer layer bonded to the inner core, wherein the outer layer comprises a metal oxide;
a first metal deposited on the outer layer, wherein the first metal is an IUPAC Group 8-10 metal; and
a second metal deposited on the outer layer, wherein the second metal is an IUPAC Group 11 or Group 14 metal;
wherein the catalyst has an accessibility index (AI) between 3 and 500, or a void space index (VSI) between 0 and 1, or both an AI between 3 and 500 and a VSI between 0 and 1;
thereby creating an ethylene product stream.

12. The process of claim 11 wherein the catalyst has been treated with an alkali metal to reduce the acidity of the catalyst.

13. The process of claim 11 wherein the selective hydrogenation conditions include pressures between 100 kPa and 14.0 MPa.

14. The process of claim 13 wherein the selective hydrogenation conditions include pressures between 500 kPa and 10.0 MPa.

15. The process of claim 14 wherein the selective hydrogenation conditions include pressures between 800 kPa and 7.0 MPa.

16. The process of claim 11 wherein the selective hydrogenation conditions include a temperature of 10° C. to 300° C.

17. The process of claim 16 wherein the selective hydrogenation conditions include a temperature of 30° C. to 200° C.

18. The process of claim 1 wherein the selective hydrogenation conditions include a hydrogen to acetylene/diolefin molar ratio between 0.1 and 20.

19. The process of claim 11 wherein the feedstream comprising ethylene and acetylene includes amounts of carbon monoxide (CO) between 0.01 and 10 ppm by volume.

20. The process of claim 11 further comprising passing the ethane/ethylene stream to an ethane/ethylene splitter, thereby creating an ethylene product stream.
